(19) United States
(12) Patent Application Publication
(10) Pub. No.: US 2002/0016561 A1
(43) Pub. Date: Feb. 7, 2002

(54) Head and neck support device

(76) Inventor: Corne Isak Prinsloo, Gautenq (ZA)

Correspondence Address: NATH & ASSOCIATES PLLC, 6th Floor, 1030 15th Street NW, Washington, DC, 20005, US

(21) Appl. No.: 09/844,431

(22) Filed: Apr. 30, 2001

(30) Foreign Application Priority Data
May 2, 2000 (ZA) 2000/2129

(51) Int. Cl. A61F 5/00

(52) U.S. Cl. 602/17

(57) ABSTRACT

A head and neck support device 10 comprises an elongate support 14. A torso harness 12 is provided towards one end of the support. A head stabilizing assembly 16 provided towards another end comprises a transverse member 26 which, in use, extends transversely over a patient 50. The transverse member suspends a head suspending member 30 which, in use, suspends a head of the patient. The device enables control of longitudinal pressure on the neck of the patient and allows normal and natural rotation of the head.

### FIELD OF THE INVENTION

THIS invention relates to a head and neck support device and more particularly, but not exclusively, to a head and neck support device for use by cerebral palsy patients.

### BACKGROUND TO THE INVENTION

People with neck dysfunctions, and especially people who suffer from cerebral palsy, need additional support to help keep their heads in an upright position. Known head support harnesses do not always provide adequate adjustability, comfort and facilitation for rehabilitation for use by different patients.

For example, in U.S. Pat. No. 5,242,377 to Boughner there is disclosed a tethered medical restrain device. The device comprises a flexible tether which is sheathed by a headband to be slidable relative thereto. The headband in use, grasps the head of a patient and the tether is anchored at a position behind the head. By leaning forward, the patient tightens the tether and is then able to rotate the head due to the aforementioned relative movement between the headband and tether.

As a result of the action of leaning forward, the neck may bulge forwardly which will cause the head to fall backwardly which is highly undesirable. Furthermore, if tension is not maintained in the tether, the head might tilt forward leftwardly or forward rightwardly. Hence, the support is not stable in use and is not suitable for many applications. Furthermore, the leaning forward action and friction between the tether and headband are not natural and may cause severe problems for patients of the aforementioned kind and may inhibit proper rehabilitation.

### OBJECT OF THE INVENTION

It is an object of this invention to provide a head and neck support device with which the applicant believes the aforementioned disadvantages may at least be alleviated.

### SUMMARY OF THE INVENTION

According to the invention there is provided a head and neck support device for a patient comprising:

an elongate support;

a torso harness mounted on the support; and

a head stabilizing assembly suspended from a suspension member which, in use, extends over a head of the patient.

The head stabilizing assembly may comprise a head suspending means.

The head suspending means is preferably mounted for selective adjustment on the suspension member which extends transversely to the support and which in turn is mounted for selective adjustment on the support.

A pivot may be provided between the suspension member and the suspending means. The pivot may comprise a ball and socket joint provided between the suspension member and a suspension bow suspending the suspending means.

The suspending means may comprise an adjustable loop for removably receiving and holding the head of the patient.

The torso harness may be mounted for selective adjustment along at least part of the length of the elongate support.

The torso harness is preferably mounted towards a first end of the elongate support and the head stabilizing assembly is preferably mounted towards a second end of the elongate support.

The torso harness may comprise a pair of shoulder yokes and a strap. The shoulder yokes and strap may be separately adjustable along at least part of the length of the support.

The torso harness may further comprise a first cushion and a second cushion may be provided towards the first end of the support.

Also included within the scope of the present invention is a head stabilizing assembly comprising a head arresting means and a pivot for connecting the arresting means to a suspension member.

### BRIEF DESCRIPTION OF THE ACCOMPANYING DIAGRAMS

The invention will now further be described, by way of example only, with reference to the accompanying diagrams, wherein:

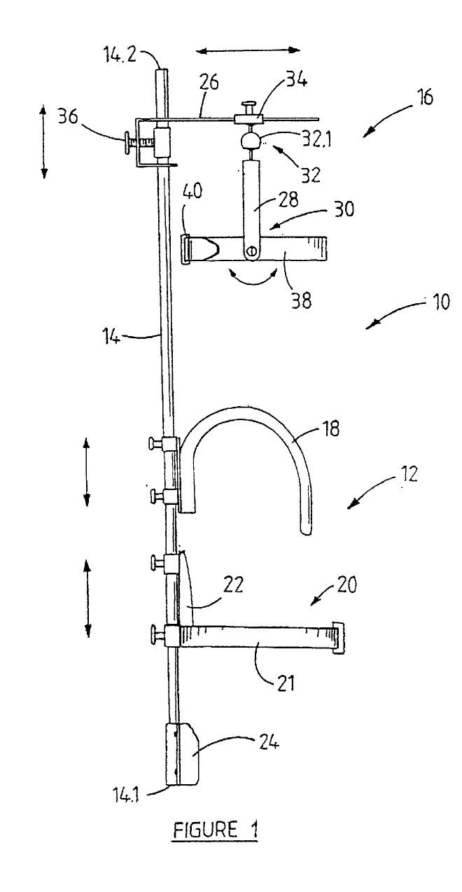

FIG. 1 is a side view of a head and neck support device according to the invention;

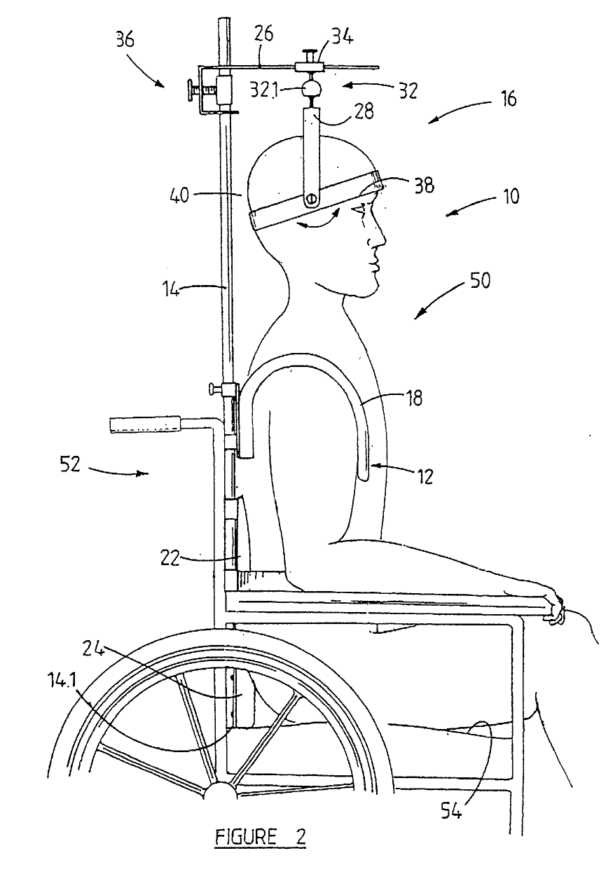

FIG. 2 is a diagrammatic side view of the device in use; and

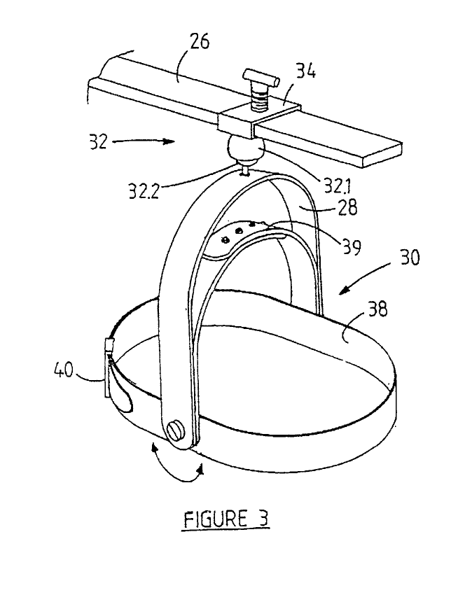

FIG. 3 is a diagrammatic perspective view of a head harness forming part of the head and neck support device of FIG. 1.

### DESCRIPTION OF A PREFERRED EMBODIMENT OF THE INVENTION

Referring to FIG. 1, a head and neck support device according to the invention is generally indicated by reference numeral 10.

The device 10 comprises a torso harness 12, an elongate support 14 having opposed ends 14.1 and 14.2 and an adjustable head stabilizing assembly 16.

The torso harness 12 comprises a shoulder yoke assembly comprising two spaced shoulder yokes 18 and a torso strap assembly 20 comprising a strap 21. The shoulder yoke assembly and torso strap assembly 20 are mounted on the elongate support 14 to be separately or collectively adjustable along the length thereof.

A first cushion 22 forms part of the strap assembly. A second cushion 24 is mounted towards the end 14.1 of the elongate support.

The adjustable head stabilizing assembly 16 comprises a lateral suspension bar 26. A suspension bow 28 for an adjustable head harness 30 depends from a ball and socket joint 32. The adjustable head harness 30 is suspended by opposite ends of the suspension bow 28. A socket 32.1 of the joint is fast with a brace 34 on the bar 26. A ball 32.2 of the joint is freely moveable in the socket. The brace is selectively adjustable on the bar 26 in direction A and the assembly 16 is selectively adjustable on elongate support 14 in direction B.

The head harness 30 comprises a loop 38 which is made of a suitable elongate flexible sheet material section. Overlapping ends of the loop 38 are releasably secured together with a loop clamp 40.

A first or proximal end of the suspension bar 26 is bent to provide a U-shaped formation. The elongate support 14 extends through apertures (not shown) in opposite legs of the U-shaped formation of the bar 26.

The first end of the lateral support bar 26 is secured to the elongate support 14 with a releasably securable clamp 36 such that the suspension bar 26 extends substantially normally from the elongate support 14. The releasably securable clamp 36 allows for the suspension bar 26 to be releasably securable anywhere along at least part of a length of the elongate support 14.

As shown in FIG. 2, in use the torso harness 12 is secured to the torso of a patient 50. The shoulder yokes 18 are located over the shoulders of the patient and the torso strap 21 is strapped around the waist of the patient. When secured in this way, the patient's back is supported against the cushion 22. With the patient in a wheelchair 52, end 14.1 of support 14 rests on a seat 54 of the chair and cushion 24 abuts against a lower back region of the patient 50.

The loop clamp 40 of head harness 30 is released to allow the overlapping ends of the loop 38 to be freely adjustable.

The first end of the suspension bar 26 is moved up or down along the elongate support 14 and the brace 34 is moved up or down the suspension bar 26 until the loop 38 locates in position around the patient's head, to grasp and hold the head of the patient.

The clamp 36 and brace 34 are tightened and in this way both the suspension bar 26 and adjustable head harness 16 are secured in place.

The overlapping ends of the loop 38 are now adjusted by sliding the overlapping ends of the loop 38 over each other until the loop fits comfortably around and holds the patient's head. The clamp 40 is closed to secure the overlapping ends of the loop 38 in position.

Hence, the device according to the invention suspends the head in use, so that it would not tilt and the pressure applied by the head on the neck is adjustable by selecting a suitable position for member 26 along support 14, Furthermore, the position of harness 30 on member 26 is also selectable, so that the patient is enabled to rotate his head normally.

It will be appreciated that there are many variations in detail on the device according to the invention without departing from the scope and spirit of the appended claims.

1. A head and neck support device for a patient comprising:
an elongate support;
a torso harness mounted on the support; and
a head stabilizing assembly suspended from a suspension member which, in use, extends over a head of the patient.

2. A device as claimed in claim 1 wherein the head stabilizing assembly comprises a head suspending means.

3. A device as claimed in claim 2 wherein the head suspending means is mounted for selective adjustment on the suspension member which extends transversely to the support and which is mounted for selective adjustment on the support.

4. A device as claimed in claim 2 wherein a pivot is provided between the suspension member and the suspending means.

5. A device as claimed in claim 4 wherein the pivot comprises a ball and socket joint provided between the suspension member and a suspension bow suspending the suspending means.

6. A device as claimed in claim 2 wherein the suspending means comprises an adjustable loop for removably receiving and holding the head of the patient.

7. A device as claimed in claim 1 wherein the torso harness is mounted for selective adjustment along at least part of the length of the elongate support.

8. A device as claimed in claim 1 wherein the torso harness is mounted towards a first end of the elongate support and the head stabilizing assembly is mounted towards a second end of the elongate support.

9. A device as claimed in claim 1 wherein the torso harness comprises a pair of shoulder yokes and a strap.

10. A device as claimed in claim 9 wherein the shoulder yokes and strap are separately adjustable along at least part of the length of the support.

11. A device as claimed in claim 1 wherein the torso harness comprises a first cushion.

12. A device as claimed in claim 11 wherein a second cushion is provided towards the first end of the support.

13. A head stabilizing assembly comprising a head arresting means and a pivot for connecting the arresting means to a suspension member.
